(19) United States
(12) Patent Application Publication
(10) Pub. No.: US 2008/0044832 A1
(43) Pub. Date: Feb. 21, 2008

(54) METHOD OF DETERMINING THE PROGNOSIS OF HUMAN CANCER

(75) Inventors: Uddhav Kelavkar, Wexford, PA (US); Rajiv Dhir, Mars, PA (US); Denise O'Keefe, Wexford, PA (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: University of Pittsburgh - Of the Commonwealth System of Higher Education, Pittsburgh, PA (US)

(21) Appl. No.: 11/737,573

(22) Filed: Apr. 19, 2007

Related U.S. Application Data
(60) Provisional application No. 60/793,100, filed on Apr. 19, 2006.

(51) Int. Cl. C12Q 1/68

(52) U.S. Cl. 435/6

(57) ABSTRACT

The invention provides methods and kits of diagnosing or determining the prognosis of a cancer in a mammal comprising determining the methylation status of the (15)-Lipoxygenase type 1 (15-LO-1) promoter in the mammal's tissue, specifically, the methylation status of the 10th CpG dinucleotide of the 15-LO-1 promoter as defined by nucleotides 727-728 of SEQ ID NO:1 in the tissue sample. The methods can include detecting the methylation status of DNA in a tissue sample, or by measuring the expression level of 15-LO-1 protein or mRNA.

### CROSS-REFERENCE TO RELATED APPLICATIONS

This patent application claims the benefit of U.S. Provisional Patent Application No. 60/793,100, filed Apr. 19, 2006, the contents of which are incorporated by reference herein.

### FEDERALLY SPONSORED RESEARCH AND DEVELOPMENT

This invention was made in part with Government support under Grant Number W81XWH awarded by the United States Department of Defense. The Government may have certain rights in this invention.

### STATEMENT REGARDING INCORPORATION-BY-REFERENCE OF MATERIAL SUBMITTED ON COMPACT DISC

Incorporated by reference in its entirety herein is a computer-readable nucleotide/amino acid sequence listing submitted concurrently herewith and identified as follows: One 7000 Byte ASCII (Text) file named “257475.5T25.TXT,” created on Apr. 19, 2007.

### BACKGROUND OF THE INVENTION

Prostate cancer is one of the leading causes of cancer deaths among men in the

United States. Although some treatments are available, such as surgery and antiestrogen therapy, curative therapies for advanced disease are lacking. Furthermore, a diagnosis of prostate cancer often requires protracted monitoring, because not all prostate abnormalities detected through common tests, such as prostate serum antigen (PSA) analysis and biopsy, are cancerous. A common course of action upon diagnosis of early prostate abnormality is “watchful waiting.” Watchful waiting requires a patient to submit to repeated monitoring tests, often over a span of years, but does not include any treatment.

Improved diagnostic methods for prostate cancer and other prostate abnormalities would reduce invasive monitoring tests, and, if such conditions are detected, reduce the duration of watchful waiting and allow treatment to begin at a less-advanced stage of disease.

### BRIEF SUMMARY OF THE INVENTION

DNA methylation is a naturally-occurring epigenetic modification that only occurs in a cytosine base followed by a guanosine base (CpG). In general, CpG hypermethylation is thought to be associated with transcriptional silencing. It is believed that methylation can result in recruitment of methylation binding proteins (MBPs) and histone deacetylation of, for example, tumor suppressor genes, inactivating them and allowing tumors to form. Surprisingly, it has been found that in the lipid peroxidating enzyme 15-LO-1, methylation of the promoter is associated with increased expression of the gene, rather than transcriptional silencing. Methylation of the 15-LO-1 gene, as well as increased expression of 15-LO-1, are now shown to be associated with cancer, such as prostate cancer, as well as some cases of prostatic intraepithelial neoplasia, a cancer-related condition.

The invention provides methods of diagnosing or determining the prognosis of a cancer in a mammal comprising determining the methylation status of the (15)-Lipoxygenase type 1 (15-LO-1) promoter in the mammal's tissue, specifically, the methylation status of the 10th CpG dinucleotide of the 15-LO-1 promoter as defined by nucleotides 727-728 of SEQ ID NO: 1 in the tissue sample (“the CpG 10 dinucleotide”).

In one aspect, the invention provides a method of diagnosing or determining the prognosis of cancer in a mammal comprising (1) providing a sample of tissue taken from a mammal; and (2) determining the methylation status of the CpG 10 dinucleotide, wherein methylation of the CpG 10 dinucleotide indicates the presence of cancer.

In another aspect, the invention provides a method of diagnosing or determining the prognosis of a cancer comprising (1) providing a sample of tissue taken from a mammal and a control sample; and (2) determining the expression level of the 15-LO-1 gene by measuring RNA or protein levels of 15-LO-1, wherein increased 15-LO-1 gene expression in the tissue sample indicates the presence of cancer.

In another aspect, the invention provides a kit for diagnosing or determining the prognosis of a cancer in a mammal comprising (1) a reagent for assaying the methylation status of the CpG 10 dinucleotide and (2) a reporter of the methylation status of the CpG 10 dinucleotide, wherein methylation of the CpG 10 dinucleotide indicates the presence of cancer.

### BRIEF DESCRIPTION OF THE SEVERAL VIEWS OF THE DRAWING(S)

FIG. 1 is a schematic illustration of the 15-LO-1 promoter sequence (SEQ ID NO: 1). The numbering of the promoter sequence is indicated by its position (reverse strand) on chromosome 17p starting at 44,991,559 nt as well as noted in parenthesis based on the translation start point (ATG) indicated by 3 asterisks (***). The HpyCH4IV site used for the cobra assay is indicated by an arrow. The CpG sites are indicated by single asterisks (*). The primer binding sites are boxed.

FIG. 2 is a sequencing analysis and methylation map of the CpG island spanning the region −217 to −474 nt within the 15-LO-1 promoter from prostate cell lines. Bisulfite converted genomic DNA from PrEC, RWPE-1, BPH-1, DU-145, LAPC-4, LNCAP, MDAPCa2b, and PC-3 cell lines was PCR amplified, TA cloned, and transformed in competent E. coli. A total of 9-10 clones were analyzed by plasmid sequencing from every cell line. Closed circle: methylated cytosine residue; open circle: unmethylated cytosine residue and boxes represent the CpG 10 dinucleotide. The numbers shown above the circles depict the cytidine residue.

FIG. 3 depicts percentage methylation of the CpG 10 dinucleotide in tissue samples from cancer tissue, PIN tissue, adjacent normal tissue, and tissue samples from healthy donors. Densitometry analyses of amplified DNA from tumor tissue (cancer, n=43), adjacent normal tissue (normal, n=37), and PIN tissue (PIN, n=1) from prostate cancer patient and normal donors (donor, n75) were digested with HpyCH₄IV and analyzed using Gel Doc video camera and Quantity One 4.1.1 software. Percentage of methylation at CpG 10 dinucleotide in prostate samples was calculated by dividing the value for the methylated band by the sum values of the lower and upper band densities. Cancer vs. donor and cancer vs. normal, P<0.05. Donor vs. PIN, P<0.1.

FIG. 4 depicts relative expression examined by relative fluorescence units (RFU's) normalized to β-actin. Results are shown for BPH1 (control), LNCaP and MDAPCa2b treated with (+) and without (−) DNA methyltransferase inhibitor (AZA, i.e., 5-aza-dC) and histone deacetylase inhibitor (TSA). P=0.001 in comparison of LNCaP cells treated vs. untreated condition. P=0.006 in comparison of MDAPCa2b cells treated vs. untreated condition.

### DETAILED DESCRIPTION OF THE INVENTION

The invention provides a method of diagnosing or determining the prognosis of a cancer in a mammal comprising (1) providing a sample of tissue taken from a mammal; and (2) determining the methylation status of the 10th CpG dinucleotide of the (15)-Lipoxygenase type 1 (15-LO-1) promoter as defined by nucleotides 727-728 of SEQ ID NO: 1 (“the CpG 10 dinucleotide”), wherein methylation of the 10th CpG dinucleotide of the 15-LO-1 promoter indicates the presence of cancer. The tissue analyzed can include tissue from a lesion; wherein methylation of the tissue sample is compared to methylation of a control sample; and wherein increased methylation in the lesional tissue indicates the presence of cancer.

The methylation status of the CpG 10 dinucleotide can be determined by any method known to one of ordinary skill in the art. For example, methylation can be determined by bisulfite treatment of DNA, reverse phase high pressure liquid chromatography (HPLC), methylation sensitive PCR (MSP), Bisulfite PCR, cloning differentially methylated sequences, Southern blot analysis, Methylated CpG island amplification (MCA), differential methylation hybridization using CpG island arrays, isolation of CpG islands using a CpG binding column, DNA-methyltransferase assay, bisulfite modification, methylation detection after restriction, methylation-sensitive restriction fingerprinting, restriction landmark genomic scanning (RLGS), and bisulfite conversion combined with bisulfite restriction analysis (COBRA). In a preferred embodiment, the method of determining methylation status is bisulfite conversion and sequencing or bisulfite conversion combined with bisulfite restriction analysis (COBRA).

If COBRA is used to determine the methylation status of the CpG 10 dinucleotide, one of ordinary skill in the art can prepare appropriate primers considering the sequence of the 15-LO-1 promoter, using any appropriate method. In one embodiment, PCR primers F15LO1 COBRA (with the sequence of 5′-TTTGTAATTTAATTTGTGAGGTTTG-3′(SEQ ID NO:2)) and R15LO1COBRA (with the sequence of 5′-CAAAAAATAAAAACCACTATCTTAAC-3′ (SEQ ID NO:3)) are used. These primers, and any primers used in the methods of the invention, can be prepared using methods that will be well known to one of ordinary skill in the art.

In another aspect, the invention provides a method of diagnosing or determining the prognosis of a cancer comprising (a) providing a sample of tissue taken from a mammal and a control sample; and (b) determining the expression level of the 15-LO-1 gene by measuring RNA or protein levels of 15-LO-1, wherein increased 15-LO-1 gene expression in the tissue sample indicates the presence of cancer.

The expression level of the 15-LO-1 gene, by RNA or protein level, can be performed by any method known to one of ordinary skill in the art. In one embodiment, quantitative RT-PCR is used. In another embodiment, immunohistochemistry with an anti-15-LO-1 antibody is used. In yet another embodiment, the method can comprise the step of determining relative 15-LO-1 activity between the tissue sample and the control sample, determination of relative activity being yet another method of measuring protein levels of 15-LO-1.

The methods of the present invention can be used to diagnose any mammal. Preferably the mammal is a human. In other embodiments, the mammal can be a non-human primate, a ruminant, a dog, a cat, a rabbit, a rodent, or another mammal.

The cancer can be any cancer in which 15-LO-1 is expressed, such as prostate cancer. The methods of the present invention can be used to diagnose cancer in a patient having a visually normal biopsy, or in a patient having a biopsy indicating an abnormality. In a preferred embodiment, the methods of the present invention are applied to patients who have been identified with prostate intraepithelial neoplasia (PIN), a proliferation of cells within the prostate that is, in some instances, a precursor to cancer. However, the methods of the present invention can also be applied to normal-appearing tissue samples to detect prostate cancer or other conditions. For example, normal appearing cells may exhibit a cancer-indicating abnormality such as methylation if they are adjacent to cancerous lesional tissue (sometimes referred to as the “field effect”). Accordingly, the presence of methylation will allow one of ordinary skill in the art to make a diagnosis of non-cancerous PIN or other non-cancerous abnormalities, as opposed to prostate cancer, using the methods of the present invention.

The invention further provides a kit for diagnosing or determining the prognosis of a cancer in a mammal comprising (1) a reagent for assaying the methylation status of the CpG 10 dinucleotide; and (2) a reporter of the methylation status of the CpG 10 dinucleotide, wherein methylation of the CpG 10 dinucleotide indicates the presence of cancer.

The reagent is a reagent for a performing a method of methylation detection. The method can be any method of methylation detection known to one of ordinary skill in the art, such as bisulfite treatment of DNA, reverse phase high pressure liquid chromatography (HPLC), methylation sensitive PCR (MSP), Bisulfite PCR, cloning differentially methylated sequences, Southern blot analysis, Methylated CpG island amplification (MCA), differential methylation hybridization using CpG island arrays, isolation of CpG islands using a CpG binding column, DNA-methyltransferase assay, bisulfite modification, methylation detection after restriction, methylation-sensitive restriction fingerprinting, restriction landmark genomic scanning (RLGS), and bisulfite conversion combined with bisulfite restriction analysis (COBRA). In a preferred embodiment, the method of methylation detection is bisulfite conversion and sequencing or bisulfite conversion combined with bisulfite restriction analysis (COBRA).

If COBRA is the method for methylation detection used, the kit can further comprise appropriate PCR primers. For example, the kit can comprise PCR primers F15LO1COBRA (SEQ ID NO:2) and R15LO1COBRA (SEQ ID NO:3). As above, such primers can be designed and prepared using any method known to one of ordinary skill in the art.

The kit of the present invention can be used in the diagnosis of any mammal. Preferably the mammal is a human. In other embodiments, the mammal can be a non-human primate, a ruminant, a dog, a cat, a rabbit, a rodent, or another mammal.

The cancer can be any cancer in which 15-LO-1 is expressed, such as prostate cancer. In a preferred embodiment, the kit as described above can be used to detect cancer in a tissue sample of a mammal who has been diagnosed with PIN. In other embodiments, the kit can be used when a biopsy appears visually normal or has an abnormality that does not appear to indicate PIN, as described above. One of ordinary skill in the art will understand that the kit of the present invention can be used on any tissue sample in which an assay for cancer is clinically indicated.

The following examples further illustrate the invention but, of course, should not be construed as in any way limiting its scope.

### EXAMPLE 1

This example demonstrates that methylation of the 15-LO-1 promoter correlates with increased expression of 15-LO-1 mRNA.

Cell lines and tissues. All cell lines were grown in a 5% CO2 incubator at 37° C. and 85% humidity. Primary prostate epithelial cells (PrEC) were maintained in PrEGM prostate epithelial cell medium (Clonetics, San Diego, Calif.). RWPE1, an immortalized normal prostate epithelial cell line, and the PCa cell lines, LNCaP, PC-3, DU145, and MDAPCa2b were obtained from the American Type Culture Collection (ATCC; Manassas, Va.) and were maintained in the recommended medium. Los Angeles Prostate Cancer-4 (LAPC-4) PCa cells were kindly provided by Dr. Robert Reiter (University of California-Los Angeles, Los Angeles, Calif.) and maintained in phenol red-free Dulbecco's modified Eagle's medium (DMEM; Invitrogen, Carlsbad, Calif.) containing 5% heat-inactivated fetal calf serum (Sigma, St. Louis, Mo.) with streptomycin-penicillin antibiotics. The benign prostatic hyperplasia cell line BPH-1 was kindly provided by Dr. Simon Hayward (Vanderbilt University, USA) and was maintained in RPMI-1640 medium supplemented with 5% FBS, HEPES buffer, and penicillin-streptomycin antibiotics. The PC₃₋₁₅LOS (15-LO-1-overexpressing) PCa cells were grown in RPMI/FBS medium containing 50 μg/ml Zeocin (Invitrogen). Kelavkar, et al., Carcinogenesis 22:1765-73 (2001).

Sections from 43 surgically resected primary prostate tumor tissues (age range 41->71 years) and 5 normal prostate specimens from cancer-free organ donors (age range 20->71 years) were obtained from the Western Pennsylvania Genitourinary Tissue Bank. Full details of tissue pathology are listed in Table 1 (below). Follow-up information was available from all but 9 patients. Follow-up information ranged from 2 weeks to 166 months, with a mean follow-up time at 91 months and a median of 90 months. Patients with disease recurrence are defined as those with a PSA value of >0.2 ng/ml post-prostatectomy or three consecutive increasing PSA values. All patients gave informed consent in accordance with the Institutional Review Board guidelines.

TABLE 1

| Clinical Parameter | Frequency (% Total) |
|---|---|
| Age Range | |
| 41-50 | 2 (4.65) |
| 51-60 | 9 (20.9) |
| 61-70 | 25 (58.1) |
| >71 | 7 (16.2) |
| Preoperative serum PSA (ng/mL) | |
| Mean | 13.8 |
| Median | 9 |
| Range | 1.8-90 |
| Pathological Stage | |
| Organ Confined (T1-T2) | 16 (37.2) |
| Capsual Penetration (T3a & T3b) | 8 (18.6) |
| Seminal Vesicle Involvement (T3c) | 5 (11.6) |
| Lymph node/Distant Metastases (N, M) | 5 (11.6) |
| Unknown | 9 (20.9) |
| Total | 43 (100) |
| Cumulative Gleason Score | |
| 6 | 11 (25.6) |
| 7 (3 + 4) | 12 (27.9) |
| 7 (4 + 3) | 12 (27.9) |
| 8-10 | 8 (18.6) |
| Total | 43 (100) |

DNA and RNA extraction. Genomic DNA was isolated from prostate cell lines with a Wizard DNA Purification System (Promega, Madison, Wis.) according to the manufacturer's instructions. The quality and integrity of the DNA was determined by the A260/280 ratio. RNA was extracted from drug treated cells using RNeasy kit (Qiagen, 2X 105 TRIzol reagent (Invitrogen, Carlsbad, Calif.). All RNA samples were treated with DNasel (Invitrogen) for 15 min at room temperature before use.

Tissue microdissection. Because of the prostate tissue heterogeneity, each tumor sample was sectioned in two consecutive 5- and 10-μM thick sections. The 5-μM slide was stained with hematoxylin and eosin (H&E), and regions of cancer, HGPIN, hyperplasia, and normal prostate were marked by a pathologist. The 10-μM section was deparaffinized, stained in Evan's Blue solution (0.5% w/v) for 10 min, followed by microdissection of marked regions under a dissecting microscope using Pinpoint resin (Zymo Research, Orange, Calif.). Briefly, the resin was applied to the marked regions, allowed to dry for 45 min and then carefully lifted off with a disposable scalpel and fine forceps to prevent cross-contamination between samples. The tissue was then incubated in proteinase K at 55° C. for 4 hr and genomic DNA purified using a DNA-binding silica column (Zymo Research).

Bisulfite conversion, cloning, sequencing from cell lines. Denatured DNA (0.5 μg) from cell lines PrEC, RWPE-1, BPH-1, DU-145, LAPC-4, LNCaP, MDAPCa2b and PC-3 was bisulfite converted using the EZ DNA Methylation Kit (Zymo Research) according to the manufacturer's directions. Briefly, the denatured DNA was incubated for 16 hr in sodium bisulfite and then desalted using DNA-binding columns. Desulphonation by incubation in sodium hydroxide was carried out within the column. The modified DNA was then eluted in 20 μl elution buffer, and 2 μl of the recovered DNA was used for PCR analysis. Bisulfite treatment converts non-methylated cytosines into uracil via deamination, which is replicated as thymidine during PCR. In contrast, 5-methyl cytosines are protected and thus identified as cytosines in the resultant PCR product. Bisulfite modified DNA was amplified by PCR with the following reaction conditions: 1X buffer (BD Biosciences; Mountain View, Calif.), 0.5 mM of each primer, 0.2 mM of each dNTP, 0.5 unit of Titanium Taq (BD Biosciences, Franklin Lakes, N.J.) and 2 μl of bisulfite-modified DNA in a final volume of 25 μl. Primers were: F15LO1 COBRA5′-TTTGTAATTTAATTTGTGAGGTTTG-3′ (SEQ ID NO:2) and R15LO1COBRA5′-CAAAAATAAAAACCACTATCTTAAC-3′ (SEQ ID NO:3) (a 258 bp PCR fragment). They are designed such that will amplify only the bisulfite converted DNA. The PCR conditions were as follows: 95° C. for 10 min for denaturation, initial 5 cycles of amplification (95° C., 30 sec; 60° C., 30 sec; 72° C., 45 sec) and then 40 cycles of amplification (95° C., 30 sec; 57° C., 30 sec; 72° C., 45 sec) with a final elongation step of 10 min at 72° C. PCR products were cloned in into pCR2.1-TOPO vector using the Invitrogen TOPO TA cloning kit (Invitrogen Life Technologies, Carlsbad, USA) and were transformed in E. coli Mach1TM-T1R cells. Blue/white screening was performed after 12 hours, and plasmids extracted and purified from ten white colonies for each cell line (except 9 colonies for DU145 cells) were sequenced by dye-terminator cycle-sequencing in both directions (both strands) on a ABI 310016-capillary instrument (Applied Biosystems, Foster City, Calif.) to identify CpG methylation.

Real-time reverse transcriptase polymerase chain reaction (RT-PCR) analysis. Total RNA from cell lines isolated by RNeasy kits was treated with DNase I (Qiagen, Valencia, Calif.), following the manufacturers protocol. RNA was quantified by spectrophotometry (Eppendorf, Germany) and its integrity was assured by analysis using BioRad Experion RNA analyzer chip (BioRad Inc., Hercules, Calif.). Real-Time quantitative PCR (qRT-PCR) reactions were performed in a 25 μl mixture containing first-strand cDNA synthesized using 1 μg of total RNA (DNase-treated) and reverse transcriptase reaction mixture. A 120-bp region of β-actin using primers 5′-CCTGGCACCCAGCACAAT-3′ (SEQ ID NO:4) and 5′-GCCGATCCACACGGAGTACT-3′ (SEQ ID NO:5) was amplified at 95° C./10m, [95° C./30s, 59° C./60s] X40 cycles using 1X SYBR Green and buffer (PE Applied Biosystems, Foster City, Calif., USA), 4 mM MgCl2, 0.2 μM of each primers (β-actin and 15-LO-1), 0.2 mM dNTPs mix and 0.025 unit of AmpliTaq Gold® thermostable DNA polymerase (Applied Biosystems, Foster City, Calif., USA). A 192 bp region of human 15-lipoxygenase-1 (15-LO-1) using primers 15-LO-15′-GACCGAGGGTTTCCTGTCTC-3′ (SEQ ID NO:6) and 5′-TGTCTCCAGCGTTGCATCC-3′ (SEQ ID NO:7) was similarly amplified (but without SYBR Green) at 95° C./3m, [95° C./30s, 58° C./60s] X 40 cycles and quantified by a TaqMan probe 5′-5HEX-CAGGCTCGGGACCAGGTTTGCCAG-BHQ2a˜5HEX-3′ (SEQ ID NO:8). Real-Time quantitation was performed using the iQ5 Real-Time PCR Detection System (BioRad, Hercules, Calif., USA).

The fluorescence threshold value was calculated using the system software. Optimization experiments showed that PCR for β-actin in triplicate were highly reproducible with a low intra-assay coefficient of variation (0.5%). Relative expression values were represented as 15-LO-1 relative fluorescence units (RFU's) normalized to β-actin from the same sample. The PCR products were subjected to 2% agarose gel electrophoresis; stained with ethidium bromide, visualized under UV illumination and quantified by scanning densitometry using the Gel Doc video camera and Quantity One 4.1.1 software (Bio-Rad). The images were divided into their respective lanes, their background subtracted, and 258 bp (uncut) and 129 bp (cut) bands were analyzed. A 100 bp ladder and the positive and negative digested controls were used for gel alignment. The normal peak density was then exported to Microsoft excel and the density values normalized for intercalation fluorescence bias by taking the natural log of the value and dividing by the number of base pairs of the fragment. The percentage of methylation was calculated by dividing the density value of the methylated band (128 bp) by the sum values of the lower and upper band densities X 100.

Expression of 15-LO-1 in malignant prostate tissue versus benign prostate disease or normal prostate tissue. Using real-time polymerase chain reaction (RT-PCR) analysis, 15-LO-1 mRNA was undetectable in the normal prostate cell lines PrEC and RWPE1. However, low levels were detected in BPH1, DU145, LAPC4, and PC3 cells while high levels of 15-LO-1 mRNA were detected in LNCaP and MDAPCa2b cell. A stably transfected PC₃₋₁₅LOS cell line served as a positive control.

DNA methylation in malignant prostate tissue versus benign prostate disease or normal prostate tissue. The second CpG island (located from −217 nt to −474 nt relative to the ATG, as studied by Liu, et al.) was evaluated for methylation in the cell lines listed above. RT-PCR was used to amplify the −217 to −474 nt region from bisulfite converted genomic DNA extracted from PrEC, RWPE-1, BPH-1, DU-145, LAPC-4, LNCaP, MDAPCa2b and PC-3 cells. The amplified DNA was cloned into TA vectors and 10 plasmids from each cell line were sequenced in both directions. The RT-PCR expression data was compared with the sequences obtained for individual cell lines.

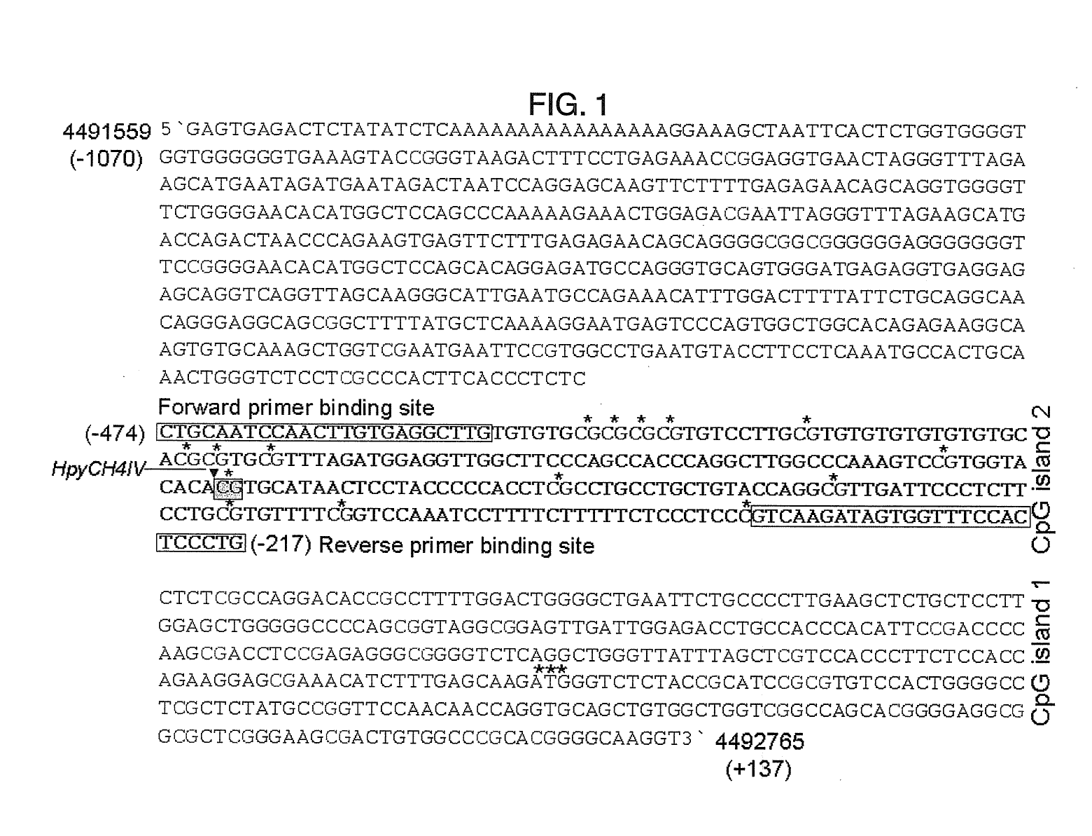

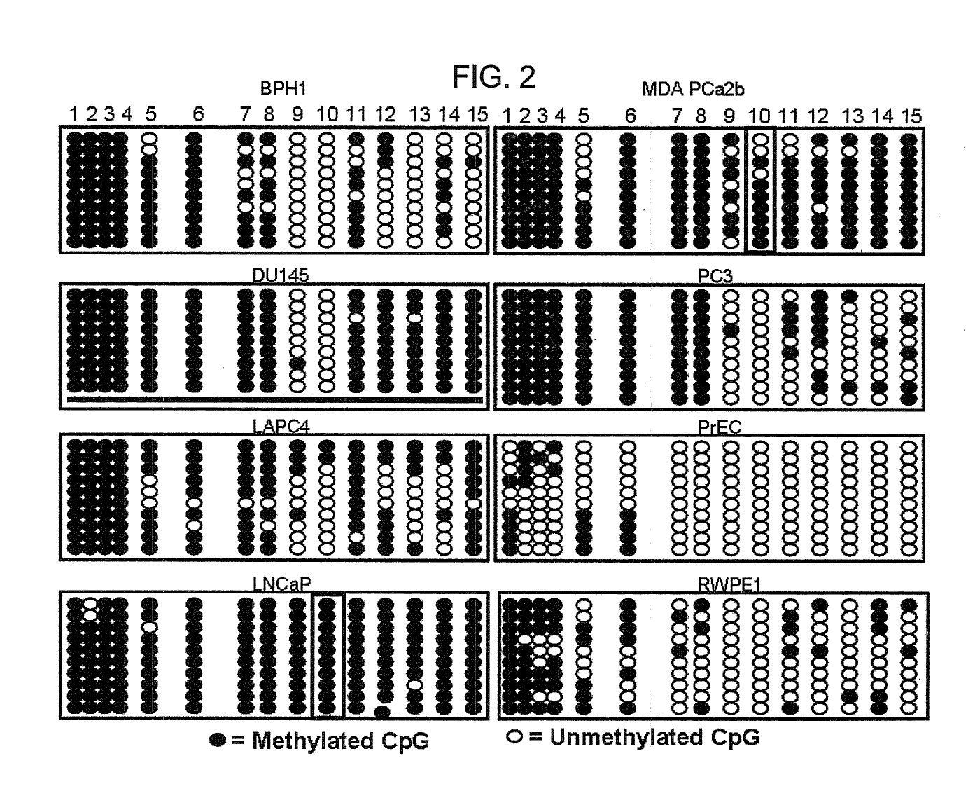

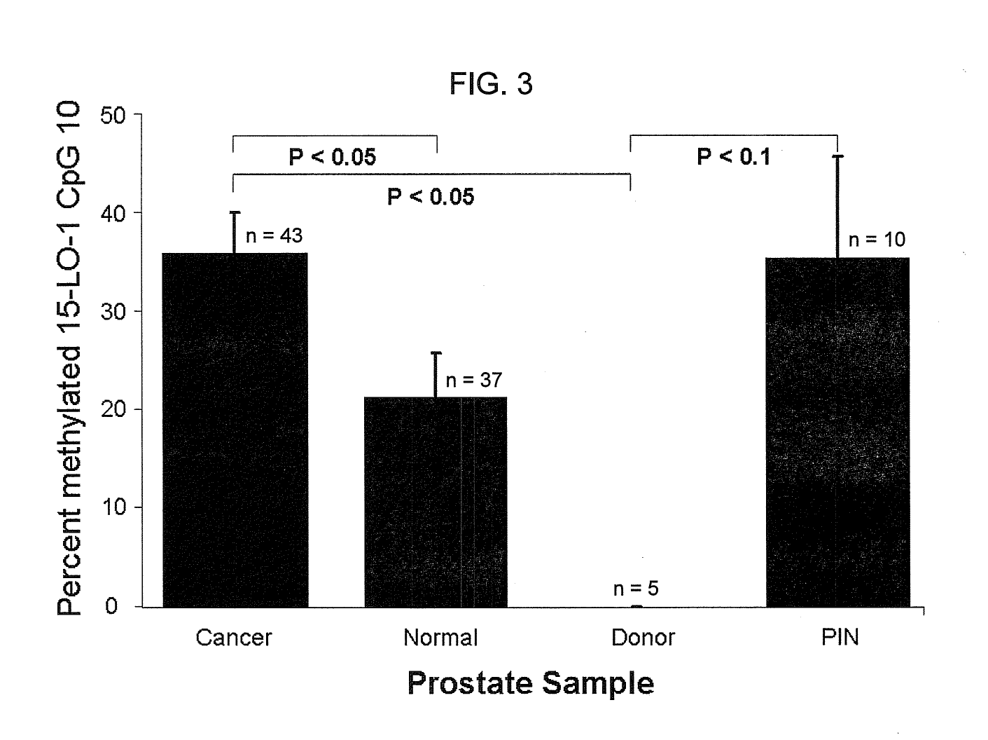

Results. From a total of 15 CpG dinucleotides in the second CpG island, methylation of the CpG 10 dinucleotide (FIG. 1 shown by *) correlates positively with 15-LO-1 mRNA expression in LNCaP and MDAPCa2b cells, as shown in the methylation map (FIGS. 2 and 3). However, methylation at other sites within the 15-LO-1 CpG island promoter region does not seem to affect transcription in prostate cancer. A similar analysis was performed on the first CpG island in the 15-LO-1 promoter, but no correlation was found.

These results show that methylation at the CpG 10 dinucleotide is correlated with a diagnosis of prostate cancer.

### EXAMPLE 2

This example demonstrates the use of combined bisulfite restriction analysis (COBRA) to evaluate the methylation status of the 15-LO-1 promoter.

Bisulfite conversion and combined bisulfite restriction analysis (COBRA). Briefly, 0.5 μg of denatured DNA from cell lines or 45 μl eluted DNA from microdissected sections were subjected to bisulfite conversion as described above. The methylation status of CpG 10 within the 15-LO-1 promoter can be determined as a percentage by subjecting the PCR product of the bisulfite modified DNA by COBRA analysis (Xiong, et al., Nucl. Acids Res., 25:2532-4 (1997)) utilizing restriction digestion with the enzyme HpyCH₄IV (New England Biolabs, Ipswich, Mass.), which cuts specifically at 5′ . . . A▾CGT . . . 3′ at CpG 10 (FIG. 1) followed by agarose gel electrophoresis. This gives a 258 bp (uncut indicating unmethylated DNA fragment) and two 129 bp (cut indicating unmethylated DNA fragment) fragments. Fifteen-LO-1 promoter fragment was PCR amplified from the bisulfite modified DNA as described above. Universally methylated DNA was bisulfite converted and included in the analysis as a control for methylation in the COBRA analyses. Normal male lymphocyte DNA, which is usually unmethylated at most promoter sites, was bisulfite converted and included as a control to represent unmethylated DNA.

Results. Bisulfite-treated genomic DNA from all the prostate cell lines was PCR amplified using F15LO1COBRA and R15LO1COBRA primers, yielding a 258 bp amplified DNA fragment. This amplified fragment was then restriction digested with HpyCH₄IV. All the normal cell lines (i.e., BPH1, RWPE1 and PrEC) showed only one uncut 258 bp band, indicating that the CpG 10 was unmethylated in normal prostate epithelial cells. However, cell lines LNCaP and MDAPCa2b showed 1 major band corresponding to the 129 bp (cut, methylated) band and a faint band at 258 bp (uncut, unmethylated). This observation confirms the results shown in the methylation map (FIG. 2). In summary, the data shows that partial promoter hypermethylation of 15-LO-1 exists in all PCa cell lines examined. The extent of methylation of the CpG 10 dinucleotide in the second CpG island of 15-LO-1 promoter corresponded with increased expression in LNCaP and MDAPCa2b cell lines. Notably, this aberrant methylation was absent in normal prostate cell lines.

### EXAMPLE 3

This example demonstrates that methylation the of 15-LO-1 promoter governs expression of 15-LO-1 in vivo.

15-LO-1 promoter methylation was assessed in 43 microdissected primary PCa specimens, 37 corresponding adjacent normal sections (taken between 2 and 17 mm from the tumor, median 10 mm), 10 adjacent HG-PIN sections and 5 normal donor prostates (Table 1).

Immunohistochemical and image cytometric analysis of prostate tissues. Sections of formalin-fixed, paraffin-embedded tissue (5 microns) were tested for the presence of 15-LO-1 [1:1600] using an avidin biotin-complex technique and steam heat-induced antigen retrieval and quantitatively examined by image cytometry as described previously (Kelavkar, et al., Carcinogenesis, 21:1777-87 (2000)).

Overall, COBRA analyses revealed that approximately 35% of cancer specimens (P<0.05 versus normal donor tissue), 20% of adjacent normal specimens, 36% adjacent HG-PIN samples (P<0.1 versus donor), and 0% normal donor specimens were positive for 15-LO-1 promoter methylation at CpG 10 (FIG. 3). Given that there was 20% methylation observed in adjacent normal specimens, this DNA was evaluated for GSTPi methylation and, it was confirmed that there was no cross-contamination between tumor samples and adjacent normal tissue that could yield a false positive result.

The methylation data was compared to the 15-LO-1 immunohistochemistry (IHC) analyses (data not shown) performed on all the tissue samples to assess whether a correlation existed between the protein expression and methylation. There was no significant correlation between overall methylation and protein expression in the tumor, Gleason score, or comparing methylation status in tumors from different pathological stages. Similarly, no significant difference in methylation is observed when cancers are compared to normal surrounding tissue, normal surrounding tissue are compared to donors or donors are compared to HGPIN. However, when donors were compared to cancer samples, there is a statistically significant difference (P<0.001) in methylation as well as in 15-LO-1 protein expression (P<0.01).

These results indicate a positive correlation between mRNA expression levels of 15-LO-1 and actual protein expression levels in prostate cell lines.

### EXAMPLE 4

This example demonstrates that DNA methylation is required for induction of 1-LO-1 mRNA.

High 15-LO-1-expressing LNCaP and MDAPCa2b cells and low 15-LO-1-expressing BPH-1 cells were treated in vitro with a combination (0.1 mM of each) of the DNA methylation inhibitor 5-aza-2′-deoxycytidine and the histone deacetylase inhibitor trichostatin-A (TSA) for 5 passages.

Combination 5-aza-2′-deoxycytidine and trichostatin A treatment. LNCaP, MDAPCa2b and BPH-1 (control) cells were plated onto several T75 cm² flasks (a total of 2×10⁷ cells each) and treated with a combination of 0.1 mM 5-aza-2′-deoxycytidine (5-aza-dC) and 0.1 mM trichostatin A (TSA) for a total of 5 passages. Untreated cells served as controls. Medium containing fresh drugs was replaced every 72 hr for the duration of the experiment. RNA was harvested after 5 passages for quantitative reverse transcription (qRT-PCR) analyses.

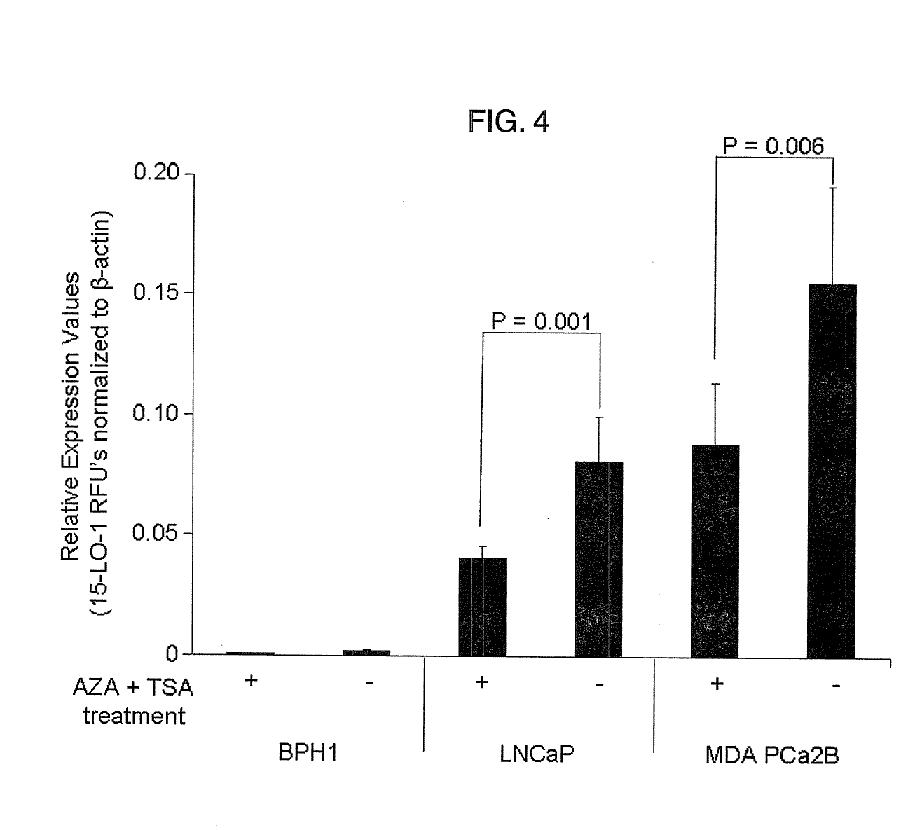

Results. Treatment of these cell lines significantly reduced the levels of 15-LO-1 mRNA. The mRNA expression was significantly reduced by 2-fold in both the cell lines with P=0.001 (LNCaP cells treated versus the untreated condition) and P=0.006 (MDAPCa2b cells treated versus the untreated condition) (FIG. 4).

These results indicate that hypomethylation of the CpG 10 in the second CpG island of the 15-LO-1 promoter is a prerequisite for the 15-LO-1 gene inactivation in prostate cells.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of diagnosing or determining the prognosis of a cancer in a mammal comprising
(1) providing a sample of tissue taken from a mammal; and
(2) determining the methylation status of the 110th CpG dinucleotide of the (15)-Lipoxygenase type 1 (15-LO-1) promoter as defined by nucleotides 727-728 of SEQ ID NO:1 in the tissue sample using a method selected from the group consisting of bisulfite treatment of DNA, reverse phase high pressure liquid chromatography (HPLC), methylation sensitive PCR (MSP), Bisulfite PCR, cloning differentially methylated sequences, Southern blot analysis, Methylated CpG island amplification (MCA), differential methylation hybridization using CpG island arrays, isolation of CpG islands using a CpG binding column, DNA-methyltransferase assay, bisulfite modification, methylation detection after restriction, methylation-sensitive restriction fingerprinting, restriction landmark genomic scanning (RLGS), and bisulfite conversion combined with bisulfite restriction analysis (COBRA);
wherein methylation of the 10th CpG dinucleotide of the 15-LO-1 promoter indicates the presence of cancer.

2. The method of claim 1, wherein the tissue analyzed includes tissue from a lesion; wherein methylation of the tissue sample is compared to methylation of a control sample; and wherein increased methylation in the lesional tissue indicates the presence of cancer.

3. The method of claim 1, wherein the method of determining methylation status is bisulfite conversion and sequencing or bisulfite conversion combined with bisulfite restriction analysis (COBRA).

4. The method of claim 3, further comprising the use of the PCR primers F16LO1 COBRA (with the sequence of 5′-TTTGTAATTTAATTTGTGAGGTTTG-3′(SEQ ID NO:2)) and R15LO1 COBRA (with the sequence of 5′-CAAAAAATAAAAACCACTATCTTAAC-3′ (SEQ ID NO:3)).

5. The method of claim 1, wherein the mammal is a human.

6. The method of claim 1, wherein the cancer is prostate cancer.

7. The method of claim 6, wherein the mammal has been diagnosed with prostate intraepithelial neoplasia (PIN).

8. The method of claim 7, wherein the tissue analyzed includes tissue from a lesion; wherein methylation of the tissue sample is compared to methylation of a control sample; and wherein increased methylation in the lesional tissue indicates the presence of PIN.

9. The method of claim 7, wherein the method of determining methylation status is bisulfite conversion and sequencing or bisulfite conversion combined with bisulfite restriction analysis (COBRA).

10. The method of claim 9, further comprising the use of the PCR primers F15LO1COBRA (with the sequence of 5′-TTTGTAATTTAATTTGTGAGGTTTG-3′ (SEQ ID NO:2)) and R15LO1COBRA (with the sequence of 5′-CAAAAAATAAAAACCACTATCTTAAC-3′(SEQ ID NO:3)).

11. The method of claim 7, wherein the mammal is a human.

12. A kit for diagnosing or determining the prognosis of a cancer in a mammal comprising:
(1) a reagent for assaying the methylation status of the 110th CpG dinucleotide of the 15-Lipoxygenase type 1 (15-LO-1) defined at nucleotides 727-728 of SEQ ID NO:1; and
(2) a reporter of the methylation status of the 110th CpG dinucleotide of the 15-LO-1 promoter,
wherein methylation of the 10th CpG dinucleotide of the 15-LO-1 promoter indicates the presence of cancer.

13. The kit of claim 12, wherein the reagent is a reagent for a performing a method of methylation detection selected from the group consisting of bisulfite treatment of DNA, reverse phase high pressure liquid chromatography (HPLC), methylation sensitive PCR (MSP), Bisulfite PCR, cloning differentially methylated sequences, Southern blot analysis, Methylated CpG island amplification (MCA), differential methylation hybridization using CpG island arrays, isolation of CpG islands using a CpG binding column, DNA-methyltransferase assay, bisulfite modification, methylation detection after restriction, methylation-sensitive restriction fingerprinting, restriction landmark genomic scanning (RLGS), and bisulfite conversion combined with bisulfite restriction analysis (COBRA).

14. The kit of claim 13, wherein the method of methylation detection is bisulfite conversion and sequencing or bisulfite conversion combined with bisulfite restriction analysis (COBRA).

15. The kit of claim 14, further comprising the PCR primers F15LO1COBRA (with the sequence of 5′-TTTGTAATTTAATTTGTGAGGTTTG-3′ (SEQ ID NO:2)) and R15LO1 COBRA (with the sequence of 5′-CAAAAAATAAAAACCACTATCTTAAC-3′ (SEQ ID NO:3)).

16. The kit of claim 12, wherein the mammal is a human.

17. The kit of claim 12, wherein the cancer is prostate cancer.

18. The kit of claim 17, wherein the mammal has been diagnosed with prostate intraepithelial neoplasia (PIN).

19. The kit of claim 18, wherein the reagent is a reagent for a performing a method of methylation detection selected from the group consisting of bisulfite treatment of DNA, reverse phase high pressure liquid chromatography (HPLC), methylation sensitive PCR (MSP), Bisulfite PCR, cloning differentially methylated sequences, Southern blot analysis, Methylated CpG island amplification (MCA), differential methylation hybridization using CpG island arrays, isolation of CpG islands using a CpG binding column, DNA-methyltransferase assay, bisulfite modification, methylation detection after restriction, methylation-sensitive restriction fingerprinting, restriction landmark genomic scanning (RLGS), and bisulfite conversion combined with bisulfite restriction analysis (COBRA).

20. The kit of claim 19, wherein the method of methylation detection is bisulfite conversion and sequencing or bisulfite conversion combined with bisulfite restriction analysis (COBRA).

21. The kit of claim 20, further comprising the PCR primers F15LO1 COBRA (with the sequence of 5′-TTTGTAATTTAATTTGTGAGGTTTG-3′ (SEQ ID NO:2)) and R15LO1COBRA (with the sequence of 5′-CAAAAAATAAAAACCACTATCTTAAC-3′(SEQ ID NO:3)).

22. The kit of claim 18, wherein the mammal is a human.

23. A method of diagnosing or determining the prognosis of a cancer comprising:
(1) providing a sample of tissue taken from a mammal and a control sample; and
(2) determining the expression level of the 15-LO-1 gene by measuring RNA or protein levels of 15-LO-1,
wherein increased 15-LO-1 gene expression in the tissue sample indicates the presence of cancer.

24. The method of claim 23, further comprising quantitative RT-PCR of 15-LO-1 RNA.

25. The method of claim 23, further comprising immunohistochemistry with an anti-15-LO-1 antibody.

26. The method of claim 23, further comprising determining relative 15-LO-1 activity between the tissue sample and the control sample.

27. The method of claim 23, wherein the mammal is a human.

28. The method of claim 23, wherein the cancer is prostate cancer.

29. The method of claim 28, wherein the mammal has been diagnosed with prostate intraepithelial neoplasia (PIN).

30. The method of claim 29, further comprising quantitative RT-PCR of 15-LO-1 RNA.

31. The method of claim 29, further comprising immunohistochemistry with an anti-15-LO-1 antibody.

32. The method of claim 29, further comprising determining relative 15-LO-1 activity between the tissue sample and the control sample.

33. The method of claim 29, wherein the mammal is a human.

34. A method of diagnosing or determining the prognosis of a cancer in a mammal comprising determining the methylation status of the (15)-Lipoxygenase type 1 (15-LO-1) promoter in the mammal's tissue.

35. The method of claim 34, wherein the 15-LO-1 promoter comprises the sequence defined at nucleotides 727-728 of SEQ ID NO: 1.

36. The method of claim 34, wherein the tissue analyzed includes tissue from a lesion and control tissue and wherein increased methylation in the lesional tissue indicates the presence of cancer.

37. The method of claim 34, further comprising bisulfite conversion and sequencing or bisulfite conversion combined with bisulfite restriction analysis (COBRA).

38. The method of claim 37, further comprising the use of the PCR primers F15LO1 COBRA (with the sequence of 5′-TTTGTAATTTAATTTGTGAGGTTTG-3′(SEQ ID NO:2)) and R15LO1COBRA (with the sequence of 5′-CAAAAAATAAAAACCACTATCTTAAC-3′(SEQ ID NO:3)).

39. The method of claim 34, wherein the mammal is a human.

40. The method of claim 34, wherein the cancer is prostate cancer.

41. A method of diagnosing or determining the prognosis of a cancer in a mammal comprising determining the methylation status of the 10th CpG dinucleotide of the 15-LO-1 promoter defined at nucleotides 727-728 of SEQ ID NO: 1 in the mammal's tissue.

42. The method of claim 41, wherein the tissue analyzed includes tissue from a lesion and control tissue and wherein increased methylation in the lesional tissue indicates the presence of cancer.

43. The method of claim 41, further comprising bisulfite conversion and sequencing or COBRA.

44. The method of claim 43, further comprising the use of the PCR primers F15LO1COBRA (with the sequence of 5′-TTTGTAATTTAATTTGTGAGGTTTG-3′(SEQ ID NO:2)) and R15LO1COBRA (with the sequence of 5′-CAAAAAATAAAAACCACTATCTTAAC-3′(SEQ ID NO:3)).

45. The method of claim 41, wherein the mammal is a human.

46. The method of claim 41, wherein the cancer is prostate cancer.

47. The method of claim 34, wherein the mammal has been diagnosed with prostate intraepithelial neoplasia (PIN).

48. The method of claim 47, wherein the 15-LO-1 promoter comprises the sequence defined at nucleotides 727-728 of SEQ ID NO: 1.

49. The method of claim 47, wherein the tissue analyzed includes tissue from a lesion and control tissue and wherein increased methylation in the lesional tissue indicates the presence of cancer.

50. The method of claim 47, further comprising bisulfite conversion and sequencing or COBRA.

51. The method of claim 50, further comprising the use of the PCR primers F15LO1COBRA (with the sequence of 5′-TTTGTAATTTAATTTGTGAGGTTTG-3′ (SEQ ID NO:2)) and R15LO1COBRA (with the sequence of 5′-CAAAAAATAAAAACCACTATCTTAAC-3′ (SEQ ID NO:3)).

52. The method of claim 47, wherein the mammal is a human.

53. The method of claim 46, wherein the mammal has been diagnosed with prostate intraepithelial neoplasia (PIN).

54. The method of claim 53, wherein the tissue analyzed includes tissue from a lesion and control tissue and wherein increased methylation in the lesional tissue indicates the presence of cancer.

55. The method of claim 53, further comprising bisulfite conversion and sequencing or COBRA.

56. The method of claim 55, further comprising the use of the PCR primers F15LO1COBRA (with the sequence of 5′-TTTGTAATTTAATTTGTGAGGTTTG-3′ (SEQ ID NO:2)) and R15LO1COBRA (with the sequence of 5′-CAAAAAATAAAAACCACTATCTTAAC-3′ (SEQ ID NO:3)).

57. The method of claim 53, wherein the mammal is a human.


SEQUENCE LISTING (8 sequences)

SEQ ID NO: 1 - DNA, 1207 bp, Homo sapiens
gagtgagactctatatctcaaaaaaaaaaaaaaaaggaaagctaattcactctggtggggtggtggggggtgaaagtaccgggtaagactttcctgagaaaccggaggtgaactagggtttagaagcatgaatagatgaatagactaatccaggagcaagttcttttgagagaacagcaggtggggttctggggaacacatggctccagcccaaaaagaaactggagacgaattagggtttagaagcatgaccagactaacccagaagtgagttctttgagagaacagcaggggcggcggggggagggggggttccggggaacacatggctccagcacaggagatgccagggtgcagtgggatgagaggtgaggagagcaggtcaggttagcaagggcattgaatgccagaaacatttggacttttattctgcaggcaacagggaggcagcggcttttatgctcaaaaggaatgagtcccagtggctggcacagagaaggcaagtgtgcaaagctggtcgaatgaattccgtggcctgaatgtaccttcctcaaatgccactgcaaactgggtctcctcgcccacttcaccctctcctgcaatccaacttgtgaggcttgtgtgtgcgcgcgcgtgtccttgcgtgtgtgtgtgtgtgcacgcgtgcgtttagatggaggttggcttcccagccacccaggcttggcccaaagtccgtggtacacacgtgcataactcctacccccacctcgcctgcctgctgtaccaggcgttgattccctcttcctgcgtgttttcggtccaaatccttttctttttctccctcccgtcaagatagtggtttccactccctgctctcgccaggacaccgccttttggactggggctgaattctgccccttgaagctctgctccttggagctgggggccccagcggtaggcggagttgattggagacctgccacccacattccgaccccaagcgacctccgagagggcggggtctcaggctgggttatttagctcgtccacccttctccaccagaaggagcgaaacatctttgagcaagatgggtctctaccgcatccgcgtgtccactggggcctcgctctatgccggttccaacaaccaggtgcagctgtggctggtcggccagcacggggaggcggcgctcgggaagcgactgtggcccgcacggggcaaggt

SEQ ID NO: 2 - DNA, 25 bp, Synthetic
tttgtaatttaatttgtgaggtttg

SEQ ID NO: 3 - DNA, 26 bp, Synthetic
caaaaaataaaaaccactatcttaac

SEQ ID NO: 4 - DNA, 18 bp, Synthetic
cctggcacccagcacaat

SEQ ID NO: 5 - DNA, 20 bp, Synthetic
gccgatccacacggagtact

SEQ ID NO: 6 - DNA, 20 bp, Synthetic
gaccgagggtttcctgtctc

SEQ ID NO: 7 - DNA, 19 bp, Synthetic
tgtctccagcgttgcatcc

SEQ ID NO: 8 - DNA, 24 bp, Synthetic
caggctcgggaccaggtttgccag